(19) United States
(12) Patent Application Publication
(10) Pub. No.: US 2009/0111133 A1
(43) Pub. Date: Apr. 30, 2009

(54) Gel Filtration Standard

(75) Inventors: Lin Gu, Lake Zurich, IL (US); Patrick A. Worklan, Abbott Park, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 11/931,778

(22) Filed: Oct. 31, 2007

(51) Int. Cl. C12Q 1/48; G01N 31/00

(52) U.S. Cl. 435/15; 436/15

(57) ABSTRACT

A gel filtration standard suitable for use as molecular weight markers for gel filtration chromatography for a mobile phase with denaturant. In one embodiment, the gel filtration standard comprises ovalbumin, myoglobin, and vitamin B₁₂. In another embodiment, the gel filtration standard comprises phosphorylase b, ovalbumin, and myoglobin along with a reducing agent, such as, for example, dithiothreitol to reduce the multiple peaks of phosphorylase b to a single peak. In a third embodiment, the gel filtration standard comprises phosphorylase b, ovalbumin, myoglobin, and vitamin B₁₂. The benefits of the gel filtration standards described herein include acceptable resolution values and usefulness in system suitability testing. The combination of markers can be used in day-to-day testing for system suitability in the denatured condition for GPC-HPLC analysis for many proteins.

### BACKGROUND OF THE INVENTION

1. Field of the Invention

This invention relates to chromatography, more particularly, a gel filtration standard suitable for determining verification of column packing and protein elution.

2. Discussion of the Art

Chromatography is a technique by which the components of a sample, carried by a liquid, are resolved on a stationary phase. Gel Permeation Chromatography (GPC), also known as size exclusion, is based on the selective permeation of soluble proteins through a column of particles of a particular size, which particles have pores of a known size. Proteins of a size larger than the pores will not enter the pores. Large proteins that do not enter the pores pass around the particles and are eluted in the void volume (Vₒ). Very small proteins and salts are retained within the particles until the total permeation volume (Vₜ) is reached. Proteins that elute between the void volume and the total permeation volume are resolved, based upon the size and shape of their molecules.

High Performance Liquid Chromatography (HPLC) systems include a high-pressure pump, an injector assembly, a signal detector, and a data collector. The systems are controlled and monitored by a computer having specialized software. The HPLC system, the analytical GPC column (stationary phase), and the mobile phase form a test system for GPC-HPLC analysis. Protein elution profile, i.e., peak retention time, peak area corresponding to peak retention time, and peak height, is continuously monitored and measured. The relative area of the major constituent peak compared to the total area of all peaks is the assessment of relative percentage purity for a sample. HPLC systems are described in Skoog and West, PRINCIPLES OF INSTRUMENTAL ANALYSIS, Second Edition, Saunders College/Holt, Rinehart and Winston (Philadelphia, Pa.; 1980), pages 690-705, incorporated herein by reference.

GPC-HPLC is generally carried out in one of two types of mobile phase, namely, native condition or denatured condition. Native condition mobile phases are typically low ionic strength buffers, such as, for example, 100 mM sodium phosphate and 150 mM sodium chloride, in pH ranges close to those of physiological conditions to preserve biological activity of the sample. The native condition mobile phase is often used for chromatographic analysis of antibody products. Denatured condition mobile phase contains denaturants, such as 0.1% SDS or 4M guanidine. The denaturants disrupt the aggregates that are formed by proteins in solution, promote uniform conformation of proteins, and reduce interaction between solutes and the matrix of the column. The denatured condition promotes an ideal separation of many proteins by means of GPC-HPLC. This denatured condition mobile phase is frequently used for chromatographic analysis of antigen products. There are no commercially available molecular weight markers for the denatured condition. There are no commercially available molecular weight markers for measuring resolution for system suitability testing for the denatured condition. When the Gel Filtration Standards from Bio-Rad Laboratories are used with a denatured mobile phase (100 mM sodium phosphate containing 0.1% SDS), the five major peaks showed above cannot be identified.

In the pharmaceutical industry, for example, because numerous tests are carried out each day in research laboratories and quality control laboratories, and because numerous GPC-HPLC tests are carried out to determine the purity of a product, it is important that the GPC-HPLC system is functioning properly on any given run. It is also important to ascertain that the GPC-HPLC system is operating in an appropriate manner and that an aged column has not degraded from the previous day's runs. The accuracy and precision of GPC-HPLC data collected rely upon an accurate and precise chromatographic system. System suitability specifications and tests to determine same are the parameters that aid in achieving accuracy and precision. System suitability tests verify the proper functioning of a GPC-HPLC system on a day-to-day basis.

In general, molecular weight markers (gel filtration standards, protein molecular weight standards) are used to calculate molecular weights of samples to monitor the progress of a chromatography run. Another important role of molecular weight markers is in the use of system suitability testing. Molecular weight markers for native condition mobile phase are commercially available. Five major protein peaks can be identified with native mobile phase for Bio-Rad Laboratories' Gel Filtration Standard. These markers/standards are thyroglobulin (Mᵣ 670,000), bovine gamma globulin (Mᵣ 158,000), chicken ovalbumin (Mᵣ 44,000), equine myoglobin (Mᵣ 17,000), and vitamin B₁₂ (Mᵣ 1,350). Mᵣ means relative molecular weight.

Most chromatographic systems can automate the measurement and reporting of resolution values for these five major peaks as system suitability testing parameter. System suitability software measures resolution between a peak and the preceding integrated peak. See FIG. 1.

The Food and Drug Administration's Center for Drug Evaluation and Research (CDER) recommends setting system suitability testing specifications, including resolution for biological components. Resolution is a measure of how well two peaks are separated. For reliable quantitation, well-separated peaks are essential. Resolution is a very useful parameter if it is believed that potential interference peak(s) may appear. For example, a combination of three molecular weight markers can be used as a system suitability reagent. The molecular weights of the three markers can be 97 kDa, 44 kDa, and 17 kDa. If the peak of a potential contaminant (88 kDa) is of concern with respect to the 44 kDa purified antigen peak in the GPC-HPLC analysis, a resolution value(s) between the molecular weight markers of the 97 kDa peak and the 44 kDa peak should be selected for monitoring. The resolution value(s) is (are) essential to ensure satisfactory separation between the 44 kDa antigenic protein and the 88 kDa contaminant to ensure that the 44 kDa antigen is pure.

The denatured condition is important in the analysis of proteins or peptides for Gel Filtration Chromatograph with Silica-Based GPC columns. The GPC-HPLC analysis for manufactured antigen products requires the use of the denatured mobile phase. Under denatured conditions, antigens do not form aggregates, and they can be eluted in a single symmetrical peak, with the result that denatured antigens can be analyzed more accurately than can native antigens in a chromatographic analysis. Under denaturing conditions, denaturants can disrupt the tertiary and quaternary structure of a protein, thereby resulting in multiple peaks for some proteins. For example, thyroglobulin has multiple peaks in SDS mobile phase. Some proteins show a single peak, but present broad and tailing elution profile that may overlap with other protein peaks, e.g., bovine IgG in SDS mobile phase. Broad and tailing peak can reduce resolution value to an unacceptable level. A protein suitable for use as a molecular weight marker that allows for measuring resolution under denaturing conditions must have a major single, sharp, and symmetrical elution profile.

### SUMMARY OF THE INVENTION

In one aspect, this invention provides a gel filtration standard suitable for use as molecular weight markers for gel filtration chromatography for a mobile phase with denaturant.

In one embodiment, the gel filtration standard comprises ovalbumin, myoglobin, and vitamin B₁₂. In this embodiment, the concentration of ovalbumin can range from about 0.6 to about 1.2 mg/mL, the concentration of myoglobin can range from about 0.4 to about 0.8 mg/mL, and the concentration of vitamin B₁₂ can range from about 0.04 to about 0.08 mg/mL. The gel filtration standard comprising the compositions of ovalbumin, myoglobin, and vitamin B₁₂ can be used to cover the molecular weight range of from 45 kDa to 1.35 kDa. This range brackets the region for analysis. Three major sharp and symmetrical peaks can be identified on commercially available gel permeation chromatography columns.

In another embodiment, the gel filtration standard comprises phosphorylase b, ovalbumin, and myoglobin along with a reducing agent, such as, for example, dithiothreitol (hereinafter alternatively referred to as “DTT”) to reduce the multiple peaks of phosphorylase b to a single peak. When a reducing agent is used to decrease the multiple peaks of phosphorylase b to a single peak, vitamin B₁₂ cannot be used as a component of the gel filtration standard. The concentration of phosphorylase b can range from about 0.36 to about 0.72 mg/mL, the concentration of ovalbumin can range from about 0.6 to about 1.2 mg/mL, and the concentration of myoglobin can range from about 0.4 to about 0.8 mg/mL. The gel filtration standard comprising the compositions of phosphorylase b, ovalbumin, and myoglobin can be used to cover the molecular weight range of from 97 kDa to 17 kDa. This range brackets the region for analysis. Three major sharp and symmetrical peaks can be identified on commercially available gel permeation chromatography columns.

In a third embodiment, the gel filtration standard comprises phosphorylase b, ovalbumin, myoglobin, and vitamin B₁₂. For the third embodiment, a special quality of phosphorylase b is required. Phosphorylase b itself must have a major single, sharp, and symmetrical elution profile. The concentration of phosphorylase b can range from about 0.36 to about 0.72 mg/mL, the concentration of ovalbumin can range from about 0.6 to about 1.2 mg/mL, the concentration of myoglobin can range from about 0.4 to about 0.8 mg/mL, and the concentration of vitamin B₁₂ can range from about 0.04 to about 0.08 mg/mL. The gel filtration standard comprising the compositions of phosphorylase b, ovalbumin, myoglobin, and vitamin B₁₂ can be used to cover the molecular weight range of from 97 kDa to 1.35 kDa. This range brackets the region for analysis. Four major sharp and symmetrical peaks can be identified on commercially available gel permeation chromatography columns.

The benefits of the gel filtration standards described herein include acceptable resolution values and usefulness in system suitability testing. The combination of markers can be used in day-to-day testing for system suitability in the denatured condition for GPC-HPLC analysis for many proteins.

### BRIEF DESCRIPTION OF THE DRAWINGS

FIG. 1 is a chromatographic profile of Bio-Rad Laboratories Gel Filtration Standard Catalog #151-1901 on a Shodex gel permeation chromatography column

FIG. 2 is a chromatographic profile of galactosidase/ovalbumin/myoglobin/vitamin B₁₂ (GOMB) with SDS mobile phase on a TosoHaas gel permeation chromatography column.

FIG. 3 is a chromatographic profile of phosphorylase b/ovalbumin/myoglobin/vitamin B₁₂ (POMB) with SDS mobile phase on a TosoHaas gel permeation chromatography column.

FIG. 4 is a chromatographic profile of ovalbumin/myoglobin/vitamin B₁₂ (OMB) with SDS mobile phase on a Shodex gel permeation chromatography column.

FIG. 5 is a chromatographic profile of Bovine IgG/myoglobin/vitamin B₁₂ with SDS mobile phase on a Shodex gel permeation chromatography column.

FIG. 6 is a chromatographic profile of phosphorylase b/ovalbumin/myoglobin/dithiothreitol (POMDTT) with SDS mobile phase on a Shodex gel permeation chromatography column.

FIG. 7 is a chromatographic profile of phosphorylase b/ovalbumin/myoglobin/vitamin B₁₂/dithiothreitol (POMBDTT) with SDS mobile phase on a Shodex gel permeation chromatography column.

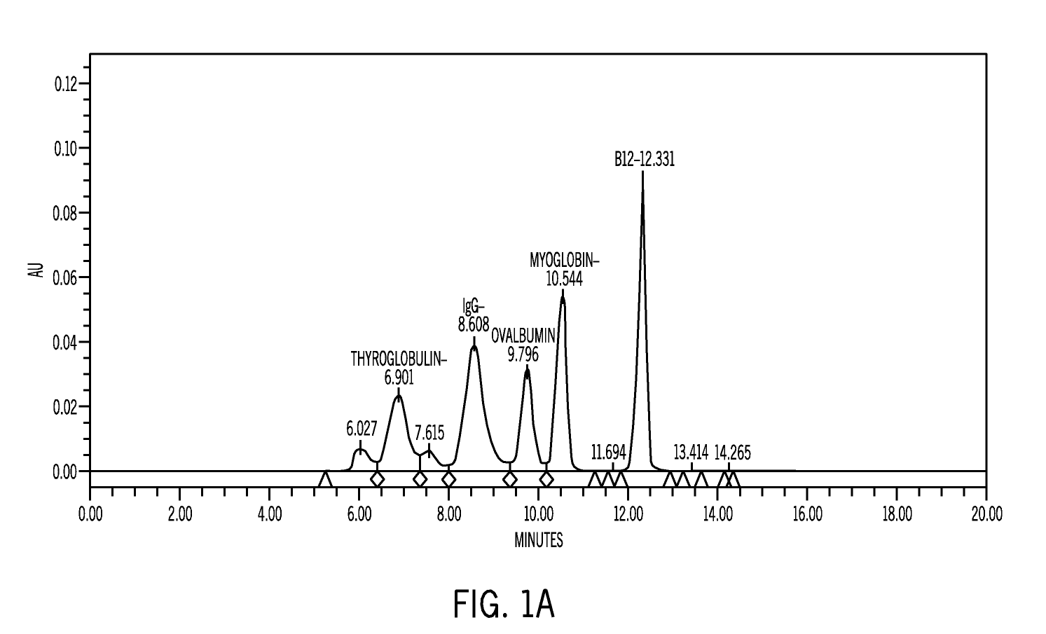

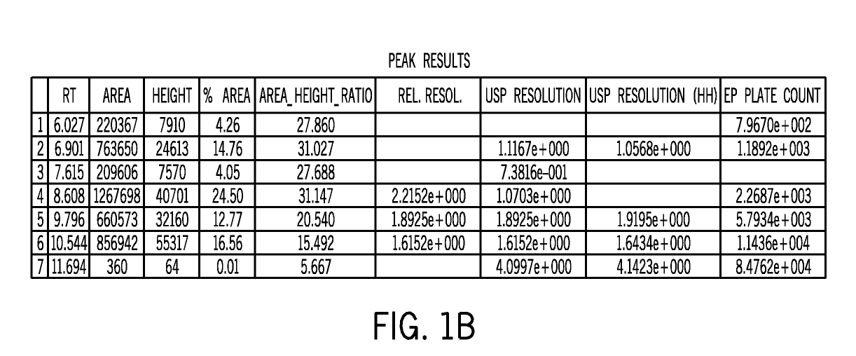

### DETAILED DESCRIPTION

As used herein, the expression “stationary phase” means beads of a porous polymeric material that readily absorbs water (and in some instances, other solvents) and swells as a consequence. The resulting solid contains a large volume of solvent held in the interstices of the polymeric network. As used herein, the expression “mobile phase” means the solvent that runs through a liquid chromatographic instrument.

As used herein, the term “SDS” means sodium dodecyl sulfate, a detergent, CAS#151-21-3, formula weight 288.38, C₁₂H₂₆O₄SNa. As used herein, the expression “system suitability testing” means testing used to verify that the resolution, detection sensitivity, and reproducibility of the chromatographic system are adequate for a chromatographic analysis to be performed. The testing is based on the concept that the equipment, electronics, analytical operation, and sample to be analyzed constitute an integral system that can be evaluated as such.

As used herein, the expression “broad peak” means a peak on a chromatogram that has eluting time greater than 1 to 2 minutes between the beginning of the peak and the end of the peak. The broad peak does not belong to the normal chromatographic profile. This undesirable phenomenon occurs when the solute experiences dilution during transit through the gel permeation chromatography column. As used herein, the expression “tailing peak” means the phenomenon In which the normal Gaussian peak has an asymmetry which is described by a so-called tailing factor, T. The tailing factor, T, is a measure of peak symmetry. The value of T is unity for perfectly symmetrical peaks, and becomes greater than one for asymmetric peaks (tailing peaks). The values of T increase as symmetry decreases. As peak asymmetry increases, integration precision becomes less reliable.

For pharmaceutical purposes, the USP tailing factor, T, is defined as the distance between the leading edge and tailing edge of the peak at a width of 5% of the peak height (w) divided by twice of the distance, F, between the peak maximum and the leading edge of the peak at 5% of peak height,

$$T = \frac{w}{2 \times F}$$

As used herein, the expression “peak retention time” means the time required for the maximum concentration of a solute to pass through a chromatography column. Peak retention time is the elution time in minutes of the peak, i.e., the time from injection to the apex of the peak. As used herein, the expression “peak area corresponding to peak retention time” means total area under the curve corresponding to the peak between the beginning of a peak and the end of a peak.

As used herein, the expression “matrix” means the solid portion of the stationary phase. As used herein, the expression “molecular weight marker” means gel filtration standard, protein molecular weight standard, or the like. In general, molecular weight markers are used to calculate the molecular weight of a sample and to monitor the progress of a chromatographic run. Molecular weight markers are also used as a positive control of the elution profile. Another important role of a molecular weight marker is its use in system suitability testing. As used herein, the expression “gel permeation chromatography” (GPC-HPLC), also known as size exclusion chromatography, means a high-pressure liquid chromatographic technique that separates molecules in solution according to their size.

The terms “denature”, “denatured”, “denaturing”, “denaturant”, “denaturation”, and the like, refer to the alteration of the structure of a protein to such an extent that some of the original properties of the protein are diminished or eliminated, such as, for example, the protein will be less able or unable to carry out its cellular function.

As used herein, an expression of the type “protein 1/protein 2/protein 3”, “protein 1/protein 2/protein 3/protein 4”, and the like, mean a mixture of the designated protein 1, protein 2, and protein 3, a mixture of the designated protein 1, protein 2, protein 3, and protein 4, etc.

In using one embodiment of the gel filtration standard described herein, for a column for GPC-HPLC having the dimensions 300 mm×7.8 mm, an injection (25 μL) contains a mixture of three molecular weight markers, namely, ovalbumin, myoglobin, and vitamin B₁₂. For the aforementioned column, in the case of an injection (25 μL), the concentration of ovalbumin can range from about 0.6 to about 1.2 mg/mL, the concentration of myoglobin can range from about 0.4 to about 0.8 mg/mL, and the concentration of vitamin B₁₂ can range from about 0.04 to about 0.08 mg/mL. For a variety of commercially available gel permeation chromatography columns for system suitability testing, an optimal ratio, based on weight, for the mixture of the proteins ovalbumin, myoglobin, and vitamin B₁₂ in the injection for providing appropriate peak heights is 15 parts by weight ovalbumin to 10 parts by weight myoglobin to 1 part by weight vitamin B₁₂, respectively. The ratio, based on weight, for the mixture of the proteins ovalbumin, myoglobin, and vitamin B₁₂ in the injection for providing appropriate peak heights can range from 15±33% parts by weight ovalbumin to 10±33% parts by weight myoglobin to 1±33% part by weight vitamin B₁₂. For a column for GPC-HPLC having the dimensions 300 mm×7.8 mm, the weight of each of the proteins can be increased or decreased proportionally based on a desired absorbance reading at 280 nm. For a smaller or a larger column, the weight of each component can be increased or decreased proportionally based on a desired absorbance reading at 280 nm, and, in addition, taking the size of the column into account. It is preferred that the injection volume of molecular weight markers be substantially the same as the injection volume of the sample to be tested.

Each molecular weight marker can be prepared first as an individual stock solution from a solid form of the protein. For preparing the individual stock solutions, a suitable concentration for each component is 10 mg/mL of ovalbumin in 0.1% SDS, 10 mg/mL of myoglobin in 0.1% SDS, and 2 mg/mL of vitamin B₁₂ in 0.1% SDS. For example, in order to prepare the stock solution of ovalbumin, ovalbumin (100 mg) can be dissolved in 0.1% SDS (10 mL) and mixed by a vortexer. The stock solution can be filtered with 0.22 to 0.45 micron syringe filter and distributed into small aliquots (e.g., 500 μL to 1 mL). The small aliquots of stock solution can be stored at a temperature of −70° C. for up to one year, and possibly longer. It is preferred that after thawing, each aliquot of stock solution be used completely, but if only a portion of the aliquot is used, the remainder of the unused aliquot should be discarded.

For preparing ready-to-use solutions for molecular weight markers, the individual stock solutions are thawed and combined. For example, ovalbumin stock solution (480 μL, 10 mg/mL), myoglobin stock solution (320 μL, 10 mg/mL), and vitamin B₁₂ stock solution (160 μL, 2 mg/mL) can be combined in a denatured mobile phase (7040 μL, 100 mM sodium phosphate buffer, 0.1% SDS) in a tube with mixing, then divided into aliquots, e.g., 250 to 500 μL aliquots. For ready-to-use molecular weight markers, the preferred concentration of ovalbumin is 0.6 mg/mL, the preferred concentration of myoglobin is 0.4 mg/mL, and the preferred concentration of vitamin B₁₂ is 0.04 mg/mL. The ready-to-use molecular weight markers can be used immediately or can be divided into aliquots and stored at −70° C. for up to 3 to 6 months from the date of preparation, and possibly longer. It is preferred that each aliquot of the ready-to-use molecular weight markers be used completely after thawing, but if only a portion of the aliquot is used, the remainder of the unused aliquot should be discarded.

Alternatively, both the individual proteins and the mixture of the mixtures can be lyophilized according to procedures well-known to those having ordinary skill in the art.

In still another alternative, the ovalbumin stock solution, the myoglobin stock solution, and the vitamin B₁₂ stock solution can be provided in a kit. The kit can contain instructions to prepare the ready-to-use version of the molecular weight markers. The instructions can contain details of the recommended ratios for preparing the mixture, the recommend volumes to be mixed, recommended conditions for storage, final concentrations of ready-to-use molecular weight markers, and information relating to stability. The kit containing the ovalbumin stock solution, the myoglobin stock solution, and the vitamin B₁₂ stock solution covers the molecular weight range of 1.35 kDa to 45 kDa. Recommended concentrations, based on weight, for the stock solutions are 10 mg/mL of ovalbumin in 0.1% SDS, 10 mg/mL of myoglobin in 0.1% SDS, and 2 mg/mL of vitamin B₁₂ in 0.1% SDS.

In using another embodiment of the gel filtration standard described herein, for a column for GPC-HPLC having the dimensions 300 mm×7.8 mm, an injection (25 μL) contains a mixture of three molecular weight markers, namely, phosphorylase b, ovalbumin, and myoglobin along with a reducing agent, e.g., dithiothreitol. An alternative reducing agent is β-mercaptoethanol. For the aforementioned column, in the case of an injection (25 μL), the concentration of phosphorylase b can range from about 0.36 to about 0.72 mg/mL, the concentration of ovalbumin can range from about 0.6 to about 1.2 mg/mL, and the concentration of myoglobin can range from about 0.4 to about 0.8 mg/mL. For a variety of commercially available gel permeation chromatography columns for system suitability testing, an optimal ratio, based on weight, for the mixture of the proteins phosphorylase b, ovalbumin, and myoglobin for providing appropriate peak heights is 9 parts by weight phosphorylase b to 15 parts by weight ovalbumin to 10 parts by weight myoglobin, respectively. The ratio, based on weight, for the mixture of the proteins phosphorylase b, ovalbumin, and myoglobin in the injection for providing appropriate peak heights can range from 9±33% parts by weight phosphorylase b to 15±33% parts by weight ovalbumin to 10±33% myoglobin. For a column for GPC-HPLC having the dimensions 300 mm×7.8 mm, the weight of each of the proteins can be increased or decreased proportionally based on a desired absorbance reading at 280 nm. For a smaller or a larger column, the weight of each component can be increased or decreased proportionally based on a desired absorbance reading at 280 nm, and, in addition, taking the size of the column into account. It is preferred that the injection volume of molecular weight markers be substantially the same as the injection volume of the sample to be tested.

Each molecular weight marker can be prepared first as an individual stock solution from a solid form of the protein. For preparing the individual stock solutions, a suitable concentration for each component is 5 mg/mL of phosphorylase b in 0.1% SDS, 10 mg/mL of ovalbumin in 0.1% SDS, and 10 mg/mL of myoglobin in 0.1% SDS. For example, in order to prepare the stock solution of phosphorylase b, phosphorylase b (10 mg) can be dissolved in 0.1% SDS (2 mL) and mixed by a vortexer. The stock solution can be filtered with 0.22 to 0.45 micron syringe filter and be divided into small aliquots (e.g., 500 μL to 1 mL). The small aliquots of stock solution can be stored at a temperature of −70° C. for up to one year, and possibly longer. It is preferred that after thawing, each aliquot of stock solution be used completely, but if only a portion of the aliquot is used, the remainder of the unused aliquot should be discarded.

For preparing ready-to-use solutions for molecular weight markers comprising phosphorylase b, ovalbumin, and myoglobin, along with a reducing agent, e.g., dithiothreitol, it is preferred that a stock solution of the reducing agent, e.g., dithiothreitol (1 M), be prepared freshly on the same day as the preparation of the molecular weight markers phosphorylase b, ovalbumin, and myoglobin. The individual stock solutions can be thawed and combined, for example, phosphorylase b (36 μL, 5 mg/mL), ovalbumin stock solution (30 μL, 10 mg/mL), myoglobin stock solution (20 μL, 10 mg/mL), and dithiothreitol stock solution (10 μL, 1M) can be combined with a denaturing mobile phase (404 μL, 100 mM sodium phosphate buffer, 0.1% SDS) in a tube and incubated at 37° C. for about 20 to 30 minutes. The ready-to-use molecular weight marker is preferably used within the same day of preparation.

Alternatively, both the individual proteins and the mixture of proteins can be lyophilized according to procedures well-known to those having ordinary skill in the art.

In still another alternative, the phosphorylase b stock solution, the ovalbumin stock solution, the myoglobin stock solution, and a reducing agent, e.g., dithithreitol, can be provided in a kit. The kit can contain instructions to prepare the ready-to-use version of the molecular weight markers. The instructions can contain details of the recommended ratios for preparing the mixture, the recommend volumes to be mixed, recommended conditions for storage, final concentrations of ready-to-use molecular weight markers, and information relating to stability. The kit containing the phosphorylase b stock solution, the ovalbumin stock solution, the myoglobin stock solution, and the reducing agent covers the molecular weight range of 17 kDa to 97 kDa. Recommended concentrations, based on weight, for the stock solutions are 5 mg/mL of phosphorylase b in 0.1% SDS, 10 mg/mL of ovalbumin in 0.1% SDS, 10 mg/mL of myoglobin in 0.1% SDS, and dithiothreitol powder.

In this embodiment, the ready-to-use molecular weight markers can be prepared without dithiothreitol. Each individual stock solution can be thawed and combined, for example, phosphorylase b (576 μL, 5 mg/mL), ovalbumin stock solution (480 μL, 10 mg/mL), and myoglobin stock solution (320 μL, 10 mg/mL) can be combined with a denatured mobile phase (6624 μL, 100 mM sodium phosphate buffer, 0.1% SDS) in a tube with mixing, and the mixture divided into aliquots, e.g., 250 to 500 μL aliquots. This ready-to-use molecular weight marker, without a reducing agent, e.g., dithiothreitol, can be used immediately by adding dithiothreitol or can be divided into aliquots and stored at −70° C. for up to 3 to 6 months, and possibly longer, from the date of preparation. After the ready-to-use aliquot is thawed, 5 μL of 1M fresh dithiothreitol should be combined with every 250 μL of the ready-to-use molecular weight solution and incubated at 37° C. for 20 to 30 minutes. It is preferred that after thawing, each aliquot of stock solution be used completely, but if only a portion of the aliquot is used, the remainder of the unused aliquot should be discarded.

If multiple peaks do not appear for a given lot of phosphorylase b, vitamin B₁₂ can be mixed with this lot of phosphorylase b, ovalbumin, and myoglobin, without a reducing agent, e.g., dithiothreitol. The concentration of phosphorylase b can range from about 0.36 to about 0.72 mg/mL, the concentration of ovalbumin can range from about 0.6 to about 1.2 mg/mL, the concentration of myoglobin can range from about 0.4 to about 0.8 mg/mL, and the concentration of vitamin B₁₂ can range from about 0.04 mg/mL to about 0.08 mg/mL.

For a variety of commercially available gel permeation chromatography columns for system suitability testing, an optimal ratio, based on weight, for the mixture of the proteins phosphorylase b, ovalbumin, myoglobin, and vitamin B₁₂ for providing appropriate peak heights is 9 parts by weight phosphorylase b to 15 parts by weight ovalbumin to 10 parts by weight myoglobin to 1±33% part by weight vitamin B₁₂, respectively. The ratio, based on weight, for the mixture of the proteins phosphorylase b, ovalbumin, myoglobin, and vitamin B₁₂ in the injection for providing appropriate peak heights can range from 9±33% parts by weight phosphorylase b to 15±33% parts by weight ovalbumin to 10±33% by weight myoglobin 1±33% part by weight vitamin B₁₂, respectively. For a column for GPC-HPLC having the dimensions 300 mm×7.8 mm, the weight of each of the proteins can be increased or decreased proportionally based on a desired absorbance reading at 280 nm. For a smaller or a larger column, the weight of each component can be increased or decreased proportionally based on a desired absorbance reading at 280 nm, and, in addition, taking the size of the column into account. It is preferred that the injection volume of molecular weight markers be substantially the same as the injection volume of the sample to be tested.

For preparing ready-to-use solutions for molecular weight markers comprising phosphorylase b, ovalbumin, myoglobin, and vitamin B₁₂. The individual stock solutions can be thawed and combined, for example, phosphorylase b (576 μL, 5 mg/mL), ovalbumin stock solution (480 μL, 10 mg/mL), myoglobin stock solution (320 μL, 10 mg/mL), and vitamin B₁₂ stock solution (160 μL, 2 mg/mL) can be combined in a denatured mobile phase (6464 μL, 100 mM sodium phosphate buffer, 0.1% SDS) in a tube with mixing, then divided into aliquots, e.g., 250 to 500 μL aliquots. For ready-to-use molecular weight markers, the preferred concentration of phosphorylase b is 0.36 mg/mL, the preferred concentration of ovalbumin is 0.6 mg/mL, the preferred concentration of myoglobin is 0.4 mg/mL, and the preferred concentration of vitamin B₁₂ is 0.04 mg/mL The ready-to-use molecular weight markers can be used immediately or can be divided into aliquots and stored at −70° C. for up to 3 to 6 months, and possibly longer, from the date of preparation. It is preferred that each aliquot of the ready-to-use molecular weight markers be used completely after thawing, but if only a portion of the aliquot is used, the remainder of the unused aliquot should be discarded.

Alternatively, the mixture can be lyophilized according to procedures well-known to those having ordinary skill in the art.

In still another alternative, the phosphorylase b stock solution, the ovalbumin stock solution, the myoglobin stock solution, and the stock solution of vitamin B₁₂ can be provided in a kit. The kit can contain instructions to prepare the ready-to-use version of the molecular weight markers. The instructions can contain details of the recommended ratios for preparing the mixture, the recommend volumes to be mixed, recommended conditions for storage, final concentrations of ready-to-use molecular weight markers, and information relating to stability. The kit containing the phosphorylase b stock solution, the ovalbumin stock solution, the myoglobin stock solution, and the vitamin B₁₂ stock solution covers the molecular weight range of 1.35 kDa to 97 kDa. Recommended concentrations, based on weight, for the stock solutions are 5 mg/mL of phosphorylase b in 0.1% SDS, 10 mg/mL of ovalbumin in 0.1% SDS, 10 mg/mL of myoglobin in 0.1% SDS, and 2 mg/mL of vitamin B₁₂ in 0.1% SDS.

### Operation

Currently, Abbott Laboratories manufactures at least 22 purified proteins that require Standard Test Procedure (STP) DFG 83 analysis using a denatured mobile phase (100 mM sodium phosphate buffer, 0.1% SDS, pH 6.8). STP DFG83 is a standard test procedure in Abbott Laboratories for Gel Permeation Chromatography by HPLC testing. These at least 22 proteins are purified recombinant antigens from either (a) different cell lines or (b) by means of different purification methods. These at least 22 antigens can be processed further to make the final product for CBER blood screening and Food and Drug Administration 510K assays. The denatured mobile phase promotes an Ideal separation of the at least 22 antigens and provides more accurate results in GPC-HPLC testing.

There is no Gel Filtration Standard commercially available for SDS mobile phase in GPC-HPLC testing. Gel Filtration Standard (GFS) Catalog #151-1901 (Bio-Rad Laboratories) is used in STP DFG83 Gel Permeation Chromatography testing for 70 antibodies with phosphate buffered saline as the mobile phase. However, the Gel Filtration Standard provided by Bio-Rad Laboratories (i.e., thyroglobulin, bovine IgG, ovalbumin, myoglobin, and vitamin B₁₂) does not exhibit five distinguishable peaks with a mobile phase containing 0.1% SDS. Because thyroglobulin appears as multiple peaks, and bovine IgG, mouse IgG, and aprotinin have broad and tailing peaks, these proteins are not considered appropriate protein markers for SDS mobile phase.

A major symmetric peak was observed from injections of multiple lots of ovalbumin (45 kDa), myoglobin (17.5 kDa), and vitamin B₁₂ (1.35 kDa). A major symmetric peak was observed from injections of one lot of galactosidase (116 kDa), phosphorylase b (97 kDa). Phosphorylase b, which is commonly used as a component for SDS-PAGE molecular weight standard, along with ovalbumin, myoglobin, galactosidase, and vitamin B₁₂ were tested in the following examples. The following non-limiting examples illustrate the invention described herein

### EXAMPLE 1

The purpose of this example is to show the feasibility of using a mixture of phophorylase b, ovalbumin, myoglobin, and vitamin B₁₂ as a system suitability reagent.

The following equipment was used in this example:

1. Photodiode array detector (Waters 2996, Waters, Milford, Mass.)

2. Separations module (Waters 2695, Waters, Milford, Mass.)

3. P1000 pipette (Pipetman®, Gilson, Inc., Middleton, Wis.)

4. P200 pipette (Pipetman®, Gilson, Inc., Middleton, Wis.)

5. P20 pipette (Pipetman®, Gilson, Inc., Middleton, Wis.)

6. P10 pipette (Pipetman®, Gilson, Inc., Middleton, Wis.)

The following gel permeation chromatography column was used in this example:

Toso-Haas G4000SWXL, 08542 B3247-83A (Waters, Milford, Mass.)

The following reagents were used in this example:
1. Ovalbumin/myoglobin/vitamin B₁₂ (OMB) ready-to-use solution
2. Phosphorylase b stock solution, 97 kDa, 10 mg/mL (according to the concentration stated on the label of the container from the vendor)
3. Ovalbumin stock solution, 45 kDa, 10 mg/mL
4. Myoglobin stock solution, 17.5 kDa, 10 mg/mL
5. Vitamin B₁₂ stock solution, 1.35 kDa, 2 mg/mL
6. Galactosidase stock solution, 116 kDa, 1 mg/mL
7. GPC column buffer (100 mm sodium phosphate buffer, 0.1% SDS, pH 6.8)
8. Sodium azide (0.05%)
9. Methanol (20%)
10. β-Mercaptoethanol, Bio-Rad Laboratories 161-0710
   The following procedure was used to carry out this example:

On the first day (day 1), phosphorylase b (5 mg/mL) was prepared by diluting phosphorylase b stock solution (10 mg/mL, 150 μL) with an equal amount of GPC column buffer (150 μL). β-mercaptoethanol (4 μL), GPC column buffer (348 μL), and phosphorylase b (5 mg/mL, 48 μL) were combined with boiling for a period of three minutes. The injection volume was 25 μL. The injection weight was 15 μg.

GPC column buffer (352 μL) and phosphorylase b (5 mg/mL, 48 μL) were combined with boiling for a period of three minutes. The injection volume was 25 μL. The injection weight was 15 μg.

GPC column buffer (352 μL) and phosphorylase b (5 mg/mL, 48 μL) were combined without boiling. The injection volume was 25 μL. The injection weight was 15 μg.

GPC column buffer (304 μL), phosphorylase b (5 mg/mL, 48 μL), ovalbumin stock solution (10 mg/mL, 24 μL), myoglobin stock solution (10 mg/mL, 16 μL), and vitamin B₁₂ stock solution (2 mg/mL, 8 μL) were combined without boiling. The injection volume was 25 μL. The injection weight of each component follows the name of the component:

| | |
|---|---|
| phosphorylase b: | 15 μg |
| ovalbumin: | 15 μg |
| myoglobin: | 10 μg |
| vitamin B₁₂: | 1 μg |

Phosphorylase b stock solution (60 μL, 10 mg/mL) was mixed with GPC column buffer (540 μL), vortexed, absorbance read at A280/A320, and concentration calculated on the basis of the A280 reading. The results of the A280 reading were as follows:

TABLE 1

| Label | | A280/A320 | |
|---|---|---|---|
| concentration | Dilution | Reading | Actual concentration |
| 10 mg/mL | 1:10 | A280: 0.053 | (0.053 × 10)/1.18 = 0.4 mg/mL |
| | | A320: 0.006 | |

Phosphorylase b (25 mg), ordered from Sigma Chemical Company, was resuspended in GPC column buffer (2.5 mL) so that the concentration expected, based on the label of the container, would be 10 mg/mL. However, the A280 reading result indicated that the actual protein concentration was about 25 times less than the concentration expected, based on the data printed on the label. Sigma Chemical Company was contacted, and it was confirmed by technical support personnel at Sigma Chemical Company that the particular lot of this phosphorylase b reagent in question was packed incorrectly, the concentration as determined by A280 and A320 was 0.4 mg/mL, and the concentration of the reagent was treated as such in further experiments.

On the day immediately succeeding day 1 (day 2), based on A280 reading results, samples of a mixture of phosphorylase b, ovalbumin, myoglobin, and vitamin B₁₂ and a mixture galactosidase, ovalbumin, myoglobin, and vitamine B₁₂ for gel permeation chromatography (GPC) were prepared as follows:

Phosphorylase b stock solution (352 μL), ovalbumin stock solution (24 μL), myoglobin stock solution (16 μL), and vitamin B₁₂ stock solution (8 μL) were combined. The injection weight of each component follows the name of the component:

| | |
|---|---|
| phosphorylase b: | 8.8 μg |
| ovalbumin: | 15 μg |
| myoglobin: | 10 μg |
| vitamin B₁₂: | 1 μg |

Ovalbumin stock solution (13.5 μL), myoglobin stock solution (9 μL), and vitamin B₁₂ stock solution (4.5 μL), GPC column buffer (108 μL), and galactosidase stock solution (90 μL) were combined. The injection volume was 25 μL. The injection weight of each component follows the name of the component:

| | |
|---|---|
| galactosidase: | 10 μg |
| ovalbumin: | 15 μg |
| myoglobin: | 10 μg |
| vitamin B₁₂: | 1 μg |

In order to operate the gel permeation chromatography column, the degasser was turned on. The plunger seal-wash pump was primed. A wet prime for all lines at 5 mL/min was performed for five minutes. The needle-wash pump was primed two to three times. The injector was purged for a minimum of six loop volumes for two to three times. The detector was turned on and one hour was allowed to pass for the Waters 2996 detector to stabilize before data was collected. The column was equilibrated for a minimum 60 minutes at a flow rate of 1.0 mL/min. General details on the operation of a GPC-HPLC column can be found in Skoog and West, PRINCIPLES OF INSTRUMENTAL ANALYSIS, Second Edition, Saunders College/Holt, Rinehart and Winston (Philadelphia, Pa.; 1980), pages 690-705, incorporated herein by reference. See also Waters 2695 Separations Module Quick Start Guide, 71500269503, Revision A, Waters, Milford, Mass., incorporated herein by reference. The following table summarizes the various samples utilized in this example.

TABLE 2

| Name of sample | |
|---|---|
| (day 1) | Identity of sample |
| Blank 1, 2, 3, 4, | Column buffer blanks, 10 μL per injection |
| 5 and 6 | |
| OMB beginning | ovalbumin (15 μg)/myoglobin (10 μg)/ |
| OMB end | vitaminB₁₂ (1 μg) standard in 0.1% |
| | SDS, 25 μL per injection |
| P not boiling | phosphorylase b in 0.1% SDS, no boiling, 25 μL per |
| | injection |
| P boiling | phosphorylase b in 0.1% SDS, boiled 3 minutes, |
| | 25 μL per injection |
| P boiling + β- | phosphorylase b in 0.1% SDS, |
| mercaptoethanol | 1% β-mercaptoethanol, boiled 3 minutes, |
| | 25 μL per injection |
| P not boiling OMB | phosphorylase b/ovalbumin/myoglobin/vitamin |
| | B₁₂ in 0.1% SDS, no boiling, 25 μL per injection |
| Name of sample | |
| (day 2) | Sample identity |
| Blank 1, 2, 3, and 4 | Column buffer blanks, 10 μL per injection |
| OMB | ovalbumin (15 μg)/myoglobin (10 μg)/vitamin |
| | B₁₂ (1 μg) standard in 0.1% SDS, |
| | 25 μL per injection |
| POMB | phosphorylase b (8.8 μg)/ovalbumin (15 μg)/ |
| | myoglobin (10 μg)/ vitamin B₁₂ (1 μg) standard |
| | in 0.1% SDS, 25 μL per injection |
| GOMB | galactosidase (10 μg)/ovalbumin (15 μg)/myoglobin |
| | (10 μg)/vitamin B₁₂ (1 μg) standard in 0.1% SDS, |
| | 25 μL per injection |

The POMB and GOMB injections of the sample set were analyzed by means of conventional integration and ApexTrack™ integration using Empower™ 2 software (Waters, Milford, Mass.). Because similar results were obtained, only the conventional Integration analysis data were used in the calculation. The following table summarizes the elution profile of phosphorylase b. Phosphorylase b was examined to determine whether phosphorylase b provided a single major symmetrical peak only.

TABLE 3

| | Retention Time | Peak Area | Peak Height |
|---|---|---|---|
| Sample | (minutes) | (microvolts) | (microvolts) |
| P not boiling | 8.155 | 31705 | 1346 |
| P boiling | 8.230 | 29916 | 1204 |
| P boiling + | 8.135 | 32948 | 1450 |
| β-mercaptoethanol | | | |

Similar elution profiles were observed from the three conditions (not boiling, boiling, boiled with β-mercaptoethanol) with the SDS mobile phase. It should be noted that only 0.6 μg was injected on account of the packing error of Sigma Chemical Company, which was described previously.

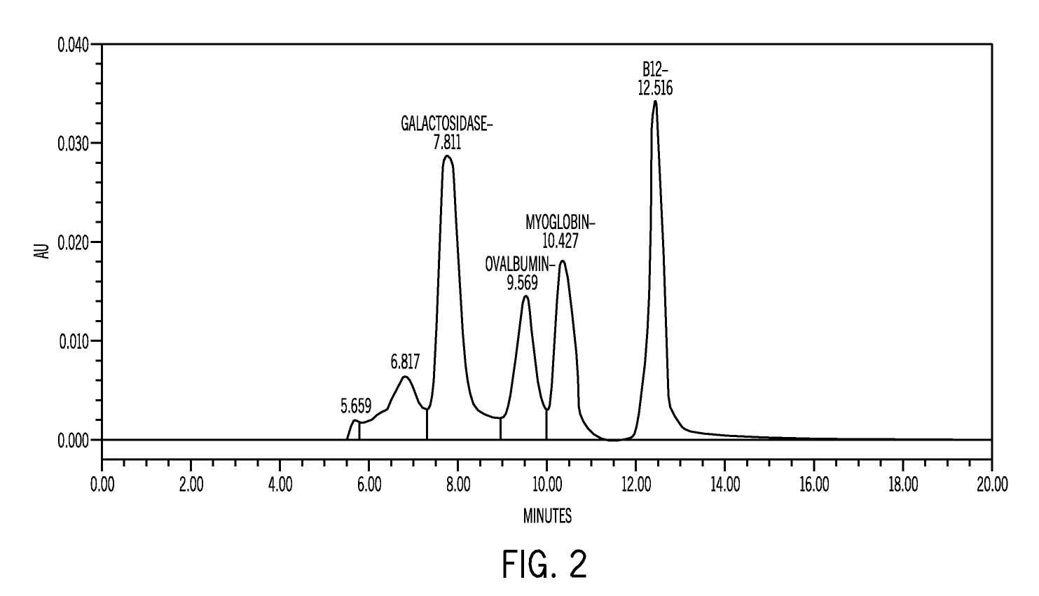

Referring now to FIG. 2, the GOMB (galactosidase/ovalbumin/myoglobin/vitamin B₁₂) sample exhibited four major peaks with the SDS mobile phase. Molecular weights were 116 kDa for galactosidase, 45 kDa for ovalbumin, 17 kDa for myoglobin, and 1.35 kDa for vitamin B₁₂. Several large aggregates were eluted from 5 to 7 minutes.

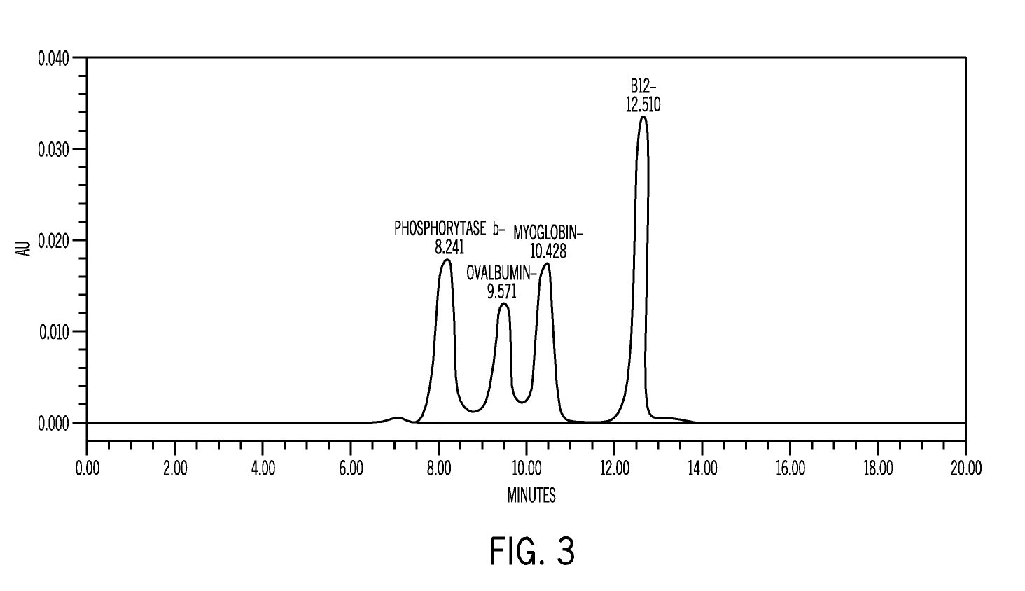

Referring now to FIG. 3, the POMB (phosphorylase b/ovalbumin/myoglobin/vitamin B₁₂) sample exhibited four major peaks with the SDS mobile phase. Molecular weights were 97 kDa for phosphorylase b, 45 kDa for ovalbumin, 17 kDa for myoglobin, and 1.35 kDa for vitamin B₁₂. Only a small aggregate was observed at about 7.5 minutes.

The following table lists the antigen codes and the antigens expected to be run in an SDS mobile phase. TABLE 4 lists molecular weights of 22 antigens requiring the SDS mobile phase.

TABLE 4

| STP DFG | | | |
|---|---|---|---|
| 83 testing | | | Molecular |
| required Code | Description | Antigen | weight (kDa) |
| 12788 | Antigen p36A HIV-2 HLH | Human Immunodeficiency Virus Type 2 Antigen from the cell line | 16 |
| | | Antigen p36A HIV-2 HLH | |
| 24472 | Antigen p41A HIV-1 HLH-O | Human Immunodeficiency Virus Type 1 Groups O Antigen from the | 16 |
| | | cell line Antigen p41A HIV-1 HLH-O | |
| 14728 | rHBe Antigen (pHBe104/XL-1) | Hepatitis B virus e Antigen from the cell line pHBe104/XL-1 | 14 |
| 66672 | Ag ADD AP24 pKRR955/KRR136 p24 | Human Immunodeficiency Virus Type 1 Antigen from the cell line | 33.5 |
| | | Ag ADD AP24 pKRR955/KRR136 p24 | |
| 92610 | Ag ADD pKRR955/KRR136 rp24 HIV-1 | Human Immunodeficiency Virus Type 1 Antigen from the cell line | 33.5 |
| | | Ag ADD pKRR955/KRR136 rp24 HIV-1 | |
| 92611 | Ag ADD pOM10/Pr361 HIV-1 rp41 | Human Immunodeficiency Virus Type 1 Antigen from the cell line | 39, 41 |
| | | Ag ADD pOM10/Pr361 HIV-1 rp41 | |
| 65438 | Ag ADD AP24 pJC104/XL-1 rCKS41 | Human Immunodeficiency Virus Type 2 Antigen from the cell line | 41, 45 |
| | | Ag ADD AP24 pJC104/XL-1 rCKS41 | |
| 92608 | Ag ADD pJC104/XL-1 rpCKS41 HIV-2 | Human Immunodeficiency Virus Type 2 Antigen from the cell line | 41, 45 |
| | | Ag ADD pJC104/XL-1 rpCKS41 HIV-2 | |
| 86928 | rHIV HLH-MO2 (Tripartite) Ag | Human Immunodeficiency Virus Antigen from cell line | 43, 46 |
| | | rHIV HLH-MO2 (Tripartite) Ag | |
| 92133 | Antigen p41A HIV-1 HLH-M | Human Immunodeficiency Virus Type 1 Groups M Antigen from cell | 43, 46 |
| | | line | |
| | | Antigen p41A HIV-1 HLH-M | |
| 52060 | Ag PHIV210/XL-1 | Human Immunodeficiency Virus Type 2 Antigen from the cell line | 59 |
| | | Ag PHIV210/XL-1 | |
| 61779 | Antigen pGO9-CKS/XL-1 | Human Immunodeficiency Virus Type 1 Groups O Antigen from the | 59.4 |
| | | cell line Antigen pGO9-CKS/XL-1 | |
| 18440 | rCMV Antigen (pCMV27/XL-1) | Cytomegalovirus Antigen from cell line rCMV Antigen (pCMV27/XL-1 | 61 |
| 19674 | rCMV Antigen (pCMV28/XL-1) | Cytomegalovirus Antigen from cell line rCMV Antigen (pCMV28/XL-1 | 61 |
| 21211 | rCMV Antigen (pCMV29/XL-1) | Cytomegalovirus Antigen from cell line rCMV Antigen (pCMV29/XL-1 | 61 |
| 23403 | Ag HC-29/XL-1; CKS-33C Sized | Hepatitis C Virus Antigen from cell line Ag HC-29/XL-1; CKS-33C | 62.3 |
| | | Sized | |
| 92609 | Ag ADD pTB319/XL-1 HIV-1 rpCKS41 | Human Immunodeficiency Virus Type 1 Antigen from cell line | 60 |
| | | Ag ADD pTB319/XL-1 HIV-1 rpCKS41 | |
| 98223 | Ag pTB319/XL-1 HIV-1 rpCKS41 sized | Human Immunodeficiency Virus Type 1 Antigen from cell line | 60 |
| | | Ag pTB319/XL-1 HIV-1 rpCKS41 sized | |
| 92372 | Antigen HC-23/XL-1 CKS-BCD | Hepatitis C Virus Antigen from cell line Antigen HC-23/XL-1 CKS-BCD | 61 |
| 60545 | Antigen pGO11-CKS/XL-1 | Human Immunodeficiency Virus type 1 Groups O Antigen from cell line | 67 |
| | | Antigen pGO11-CKS/XL-1 | |
| 24696 | Ag HC31/XL-1 CKS-33-BCD Sized | Hepatitis C Virus Antigen from cell line Ag HC31/XL-1 CKS-33-BCD | 92 |
| | | Sized | |
| 93429 | Ag HC31/XL-1; CKS-33-BCD Sized | Hepatitis C Virus Antigen from cell line Ag HC31/XL-1; CKS-33-BCD | 92 |
| | | Sized | |

Molecular weights of the 22 codes ranged from 14 kDa to 92 kDa. The OMB molecular weight ranged from 1.35 kDa to 45 kDa. In the GOMB elution profile, several large aggregate peaks were seen as compared with POMB with the SDS mobile phase, which exhibited only a small aggregate. The POMB chromatogram showed four symmetrical peaks and appeared to be a better choice for the molecular weight range for an antigen having a molecular weight of from 14 to 92 kDa than the OMB and the GOMB. The POMB appeared to be a potential candidate for molecular weight markers and system suitability reagents suitable for the SDS mobile phase for the 22 antigens in the STP DFG83 listing. In addition, the inclusion of a reducing agent does not appear to improve the characteristics of GOMB as a gel filtration standard.

### EXAMPLE 2

Aggregation peaks appeared from the chromatograms of the new lots of phosphorylase b. In this example, dithiothreitol (DTT) was used as a reducing reagent to determine whether the aggregation peaks can be eliminated.

The same equipment that was used in the previous example was used in this example. However, in this example, the following GPC column was used.

Shodex, PROTEIN KW-803

In this example, the same reagents that were used in the previous example were used in this example, with the following exceptions:
1. Bovine IgG, 2 mg/mL, Bio-Rad Laboratories, catalog #97124 was used to test the feasibility of using bovine IgG as a system suitability reagent for a denatured mobile phase.
2. Bovine serum albumin, Pierce, catalog #23210 was used as a system suitability reagent for the determination of the ratio of peak area of a chromatogram of bovine serum albumin to peak height of a chromatogram of bovine serum albumin.
3. 10% SDS was used as a denaturant to prepare system suitability reagents in a denatured condition.
4. Dithiothreitol, code 98216 was used to prepare a reducing reagent.
5. Dithiothreitol, Pierce, catalog #20290 was used to prepare a reducing reagent.
   Dithiothreitol (1M) was prepared by dissolving dithiothreitol (154 mg) in water (1 mL) and vortexing the resultant mixture. Phosphorylase b/ovalbumin/myoglobin with dithiothreitol was prepared as follows:

Phosphorylase b stock solution (360 μL), ovalbumin stock solution (24 μL), myoglobin stock solution (16 μL), dithiothreitol (1M, 8 μL) were combined. The injection volume was 25 μL. The injection weight of each component follows the name of the component:

| | |
|---|---|
| phosphorylase b: | 9 μg |
| ovalbumin: | 15 μg |
| myoglobin: | 10 μg |
| dithiothreitol: | 20 mM |

The mixture was incubated at 37° C. for 20 minutes.

Phosphorylase b/ovalbumin/myoglobin/vitamin B₁₂with dithiothreitol was prepared as follows:

Phosphorylase b stock solution (352 μL), ovalbumin stock solution (24 μL), myoglobin stock solution (16 μL), vitamin B₁₂ stock solution (8 μL), 1M dithiothreitol (8 μL) were combined. The injection volume was 25 μL. The injection weight of each component follows the name of the component:

| | |
|---|---|
| phosphorylase b: | 9 μg |
| ovalbumin: | 15 μg |
| myoglobin: | 10 μg |
| vitamin B₁₂: | 1 μg |
| dithiothreitol | 20 mM |

The mixture was incubated at 37° C. for 20 minutes.

Bovine IgG/Myoglobin/vitamin B₁₂ was prepared as follows:

Bovine IgG (80 μL), myoglobin stock solution (16 μL), vitamin B₁₂ stock solution (8 μL), and GPC column buffer (296 μL) were combined. The injection volume was 25 μL. The injection weight of each component follows the name of the component:

| | |
|---|---|
| bovine IgG: | 10 μg |
| myoglobin: | 10 μg |
| vitamin B₁₂: | 1 μg |

The same procedure that was used to run the GPC-HPLC column in the previous example was used in this example. TABLE 5 shows details of the sample injections.

TABLE 5

| Sample | |
|---|---|
| name | Sample Identity |
| Blank 1, | Column buffer blanks, 10 μL per injection |
| Blank 2, | |
| BLK | |
| POMDTT | phosphorylase b (9 μg)/ovalbumin (15 μg)/myoglobin |
| | (10 μg)/DTT (20 mM), 25 μL per injection |
| POMBDTT | phosphorylase b (9 μg)/ovalbumin (15 μg)/myoglobin |
| | (10 μg)/vitamin B₁₂ (1 μg)/DTT (20 mM), |
| | 25 μL per injection |
| OMB | ovalbumin (15 μg)/myoglobin (10 μg)/vitamin B₁₂ (1 μg), |
| | 25 μL per injection |
| BMB | bovine IgG (10 μg)/myoglobin (10 μg)/vitamin B₁₂ (1 μg), |
| | 25 μL per injection |

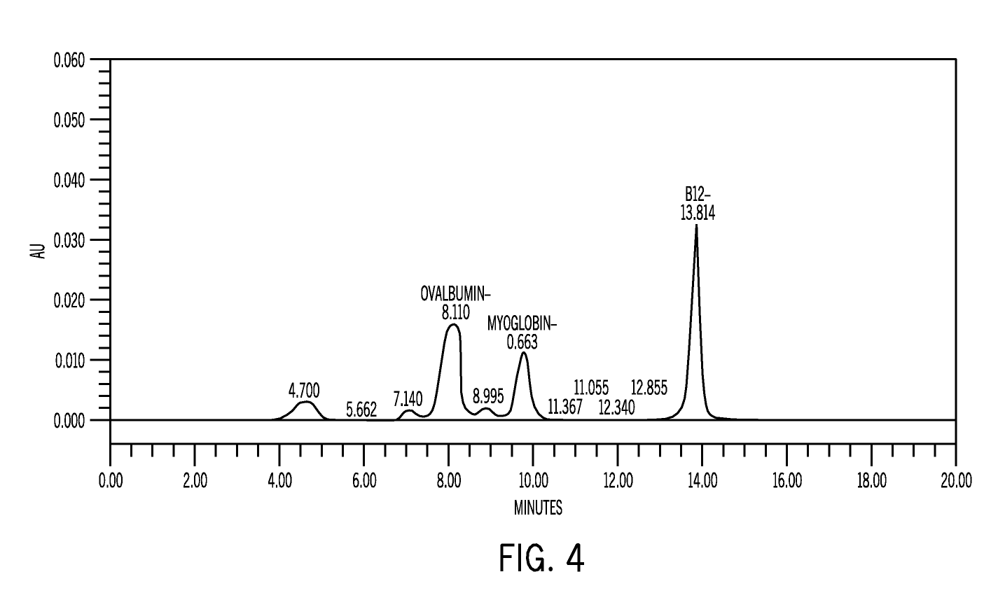

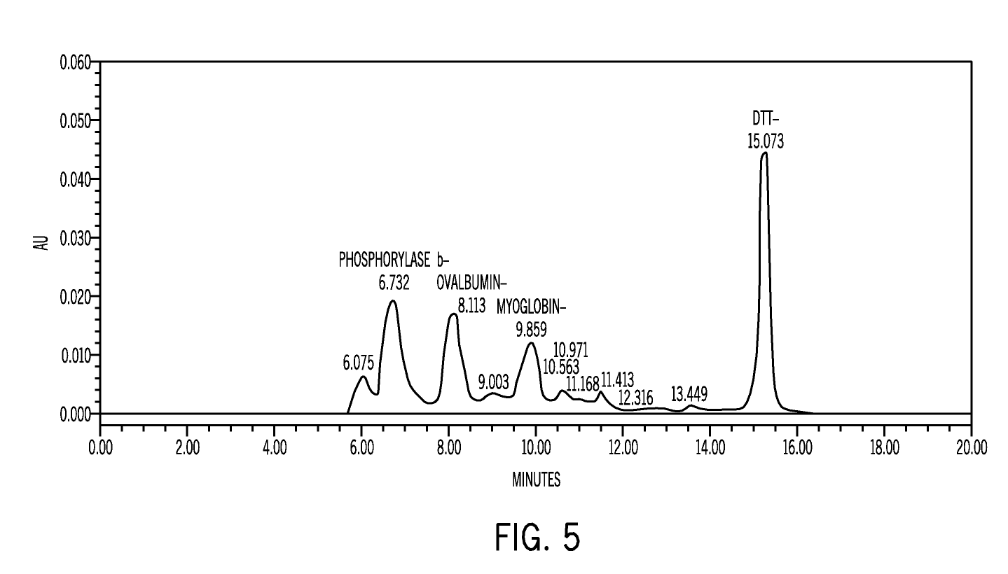

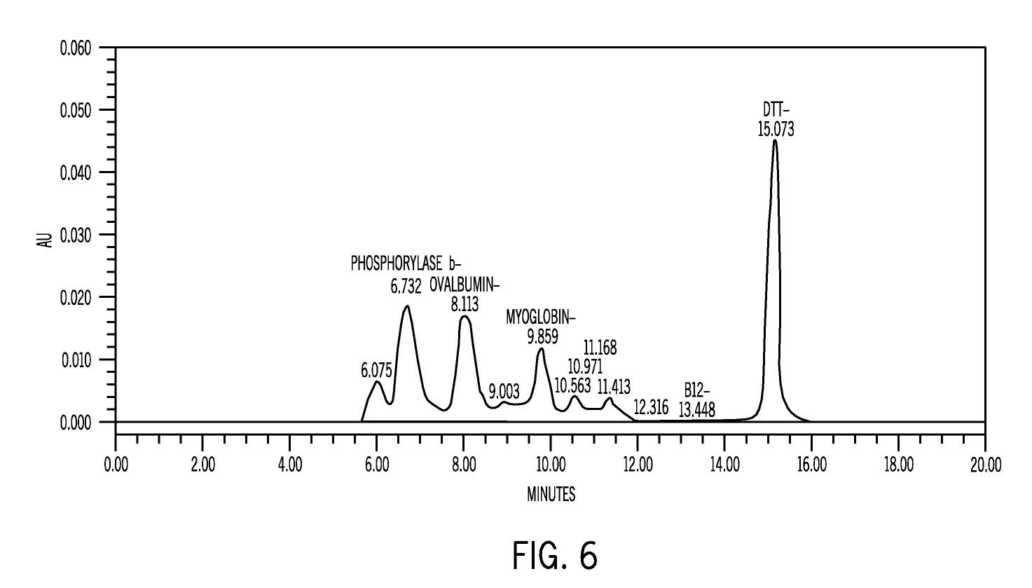

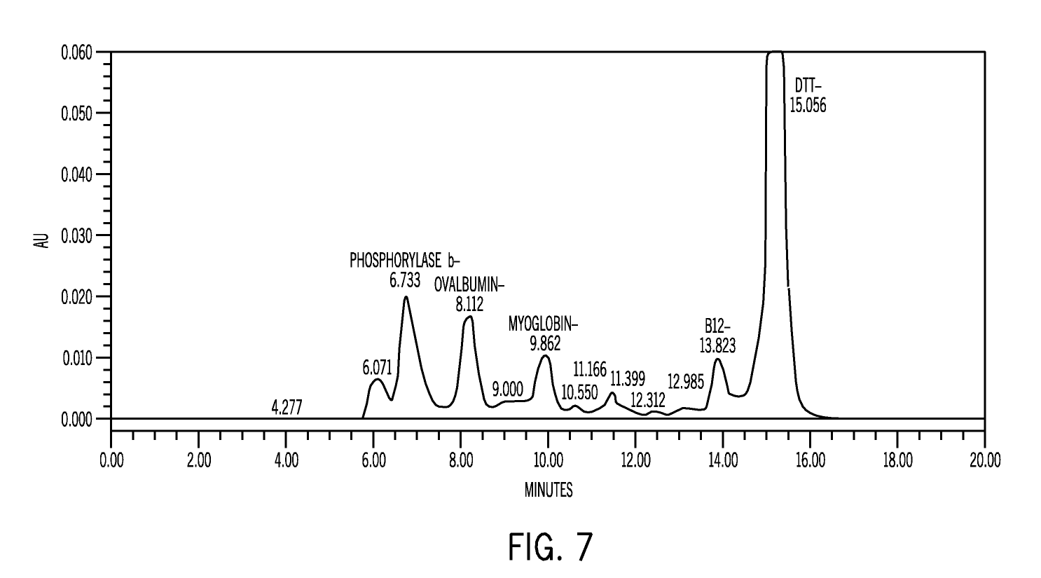

Referring now to FIG. 4, three major symmetrical peaks represent ovalbumin/myoglobin/vitamin B₁₂ (OMB) with the SDS mobile phase. Referring now to FIG. 5, the chromatographic profile of the bovine IgG/myoglobin/vitamin B₁₂ (BMB) shows a broad bovine IgG peak in the SDS mobile phase. Referring now to FIG. 6, the chromatographic profile of the reduced phosphorylase b/ovalbumin/myoglobin in 20 mM dithiothreitol (POMDTT) shows four major peaks with the SDS mobile phase. Molecular weights are 97 kDa for phosphorylase b, 45 kDa for ovalbumin, 17 kDa for myoglobin, and 166 dalton for dithiothreitol. Referring now to FIG. 7, the peaks of phosphorylase b, ovalbumin, myoglobin, vitamin B₁₂, and dithiothreitol (POMBDTT) can be identified in the SDS mobile phase. The vitamin B₁₂ peak area of POMBDTT is much smaller than the vitamin B₁₂ peak area in either OMB or BMB; the dithiothreitol peak area of POMBDTT is much larger than the dithiothreitol peaks in POMDTT. Accordingly if dithiothreitol is used as a reducing agent for phosphorylase b, vitamin B₁₂ should not be used in the gel filtration standard because dithiothreitol changes the profile of vitamin B₁₂, i.e., the profile does not accurately reflect the presence of vitamin B₁₂. The following table indicates the relative resolution of the various peak combinations.

TABLE 6

| | P/O | O/M | M/B12 | M/DTT |
|---|---|---|---|---|
| | Rel.Resol* | Rel.Resol* | Rel.Resol* | Rel.Resol* |
| POMDTT, | 1.848528 | 2.363035 | N/A | 9.244159 |
| rep 1 | | | | |
| POMDTT, | 1.854389 | 2.362160 | N/A | 9.350307 |
| rep 2 | | | | |
| POMBDTT, | 1.785308 | 2.233490 | 5.952822 | 8.592761 |
| rep 1 | | | | |
| POMBDTT, | 1.785562 | 2.244152 | 6.141976 | 8.740287 |
| rep 2 | | | | |
| OMB, rep 1 | N/A | 2.528389 | 7.137130 | N/A |
| OMB, rep 2 | N/A | 2.505244 | 7.025047 | N/A |
| BMB, rep 1 | N/A | N/A | 6.844886 | N/A |
| BMB, rep 2 | N/A | N/A | 6.880358 | N/A |

*The relative resolution = USP resolution by Waters Empower 2 software.
P/O: The peaks of phosphorylase b and ovalbumin.
O/M: The peaks of ovalbumin and myoglobin.
M/B12: The peaks of myoglobin and vitamin B₁₂.
M/DTT: The peaks of myoglobin and dithiothreitol.

The aggregation peaks were not completely eliminated through the use of dithiothreitol but were minimized. From the chromatogram of POMDTT (posphorylase b, ovalbumin, myoglobin, and dithiothreitol) the four major peaks can be identified with the SDS mobile phase. For the Shodex gel permeation chromatography column, the resolution of the phosphorylase b and ovalbumin peaks is about 1.8, on average, the resolution of the ovalbumin and myoglobin peaks is about 2.4, on average, and the resolution of the myoglobin and dithiothreitol peaks is above about 8, on average. The chromatographic profile and resolution values of POMDTT indicate that POMDTT is appropriate as system suitability reagent if only one system suitability reagent will be chosen for all the 22 antigens that require the SDS mobile phase. The data show that the dithiothreitol reduced the peak area for vitamin B₁₂ (see FIG. 7). The SDS mobile phase caused the broad bovine IgG peak (see FIG. 5). POMDTT is a good system suitability reagent when a given lot of phosphorylase b provides multiple peaks.

Various modifications and alterations of this invention will become apparent to those skilled in the art without departing from the scope and spirit of this invention, and it should be understood that this invention is not to be unduly limited to the illustrative embodiments set forth herein.

What is claimed is:

1. A gel filtration standard comprising a mixture of ovalbumin, myoglobin, and vitamin B₁₂.

2. The gel filtration standard of claim 1, wherein the mixture is lyophilized.

3. The gel filtration standard of claim 1, wherein the mixture is maintained in a frozen state.

4. The gel filtration standard of claim 1, wherein the ratio of ovalbumin to myoglobin to vitamin B₁₂ ranges from 15±33% parts by weight ovalbumin to 10±33% parts by weight myoglobin to 1±33% part by weight vitamin B₁₂.

5. A gel filtration standard comprising a mixture of phosphorylase b, ovalbumin, and myoglobin.

6. The gel filtration standard of claim 5, wherein the mixture is lyophilized.

7. The gel filtration standard of claim 5, wherein the mixture is maintained in a frozen state.

8. The gel filtration standard of claim 5, wherein the ratio of phosphorylase b to ovalbumin to myoglobin ranges from 9±33% parts by weight phosphorylase b to 15±33% parts by weight ovalbumin to 10±33% parts by weight myobglobin.

9. The gel filtration standard of claim 5, wherein the mixture further includes vitamin B₁₂, provided that the lot from which phosphorylase b is selected exhibits a single symmetrical peak only.

10. The gel filtration standard of claim 9, wherein the mixture is lyophilized.

11. The gel filtration standard of claim 9, wherein the mixture is maintained in a frozen state.

12. The gel filtration standard of claim 5, further including a reducing agent.

13. The gel filtration standard of claim 12, wherein the reducing agent is dithiothreitol.

14. A method preparing a gel filtration standard comprising the steps of:
(a) providing a stock solution of ovalbumin, a stock solution of myoglobin, and a stock solution of vitamin B₁₂; and
(b) combining said stock solutions of ovalbumin, myoglobin, and vitamin B₁₂ to form a mixture.

15. The method of claim 14, wherein the stock solution of ovalbumin, the stock solution of myoglobin, and the stock solution of vitamin B₁₂ are provided in a kit.

16. The method of claim 14, further including the step of lyophilizing the mixture.

17. A method preparing a gel filtration standard comprising the steps of:
(a) providing a stock solution of phosphorylase b, a stock solution of ovalbumin, and a stock solution of myoglobin; and
(b) combining said stock solutions of phosphorylase b, ovalbumin, and myoglobin to form a mixture.

18. The method of claim 17, wherein the stock solution of phosphorylase b, the stock solution of ovalbumin, and the stock solution of myoglobin are provided in a kit.

19. The method of claim 17, further including the step of adding a reducing agent in step (b).

21. The method of claim 19, wherein the reducing agent is dithiothreitol.

22. The method of claim 17, further including the step of lyophilizing the mixture.

23. The method of claim 17, wherein a stock solution of vitamin B₁₂ is provided and the stock solution of vitamin B₁₂ is added to the combination of the stocks solutions of phosphorylase b, ovalbumin, and myoglobin to form a mixture.

24. The method of claim 23, wherein the stock solution of phosphorylase b, the stock solution of ovalbumin, the stock solution of myoglobin, and the stock solution of vitamin B₁₂ are provided in a kit.

25. The method of claim 23, further including the step of lyophilizing the mixture.
